(19) United States
(12) Patent Application Publication
(10) Pub. No.: US 2021/0027879 A1
(43) Pub. Date: Jan. 28, 2021

(54) DEVICES, SOFTWARE, SYSTEMS, AND METHODS FOR INTRAOPERATIVELY AND POSTOPERATIVELY TRACKING THE RELATIVE POSITION BETWEEN EXTERNAL FIXATION COMPONENTS OR RINGS

(71) Applicants: Smith & Nephew, Inc., Memphis, TN (US); SMITH & NEPHEW PTE. LIMITED, Singapore (SG); Smith & Nephew Orthopaedics AG, Switzerland (CH)

(72) Inventors: Andrew P. Noblett, Cordova, TN (US); Johnny Mason, Cordova, TN (US); Charles Heotis, Cordova, TN (US)

(21) Appl. No.: 17/044,956

(22) PCT Filed: Apr. 2, 2019
(86) PCT No.: PCT/US2019/025310

§ 371 Date: Oct. 2, 2020

Related U.S. Application Data
(60) Provisional application No. 62/653,218, filed on Apr. 5, 2018.

(51) Int. Cl. G16H 20/40; G16H 40/67; A61B 17/66; A61B 34/20

(52) U.S. Cl. CPC G16H 20/40; G16H 40/67; A61B 2017/00199; A61B 34/20; A61B 17/66

(57) ABSTRACT

The present disclosure provides an intraoperative external fixation component tracking system to enable a surgeon to efficiently plan out construction of an external fixator. The intraoperative external fixation component tracking system further enables data related to the surgery, including data for determining a strut adjustment schedule for the external fixator, to be captured intraoperatively for use postoperatively. The present disclosure further provides a postoperative external fixation component tracking system to enable a patient to efficiently adjust struts of an installed external fixator. The postoperative external fixation component tracking system further enables to surgeon to remotely monitor the patients compliance with the strut adjustment schedule.

### CROSS-REFERENCE TO RELATED APPLICATIONS

This is a non-provisional of, and claims the benefit of the filing date of, pending U.S. provisional patent application number 62/653,218, filed Apr. 5, 2018, titled “Devices, Software, and Methods for Intraoperatively and Postoperatively Tracking the relative Position Between External Fixation Components or Rings,” the entirety of which application is incorporated by reference herein.

### FIELD OF THE DISCLOSURE

The present disclosure generally relates to medical devices, and more particularly, but not exclusively, relates to devices, systems, and methods for intraoperatively and postoperatively tracking the relative position between external fixation components or rings, and to devices, systems, and methods that link the intraoperative surgical procedure and the postoperative prescription software into a seamless, integrated software system.

### BACKGROUND OF THE DISCLOSURE

Orthopaedic or bone deformity correction devices or bone adjustment systems (used interchangeably herein without the intent to limit) such as, for example, hexapods, external fixators, or fixation systems are known. One well known correction device is the Taylor Spatial Frame. In use, the correction device may utilize first and second external fixation components, frames, or rings (used interchangeably herein without the intent to limit), and a plurality of adjustable bodies (e.g., typically four or six interconnecting bodies or struts). The adjustable bodies or struts (used interchangeably herein without the intent to limit) may take the form of telescopic rods so that, in use, the struts may be shortened or lengthened as required, either intraoperatively to build the correction device or postoperatively to adjust the relative position between the first and second fixation components, and hence the bones attached thereto. As a result, each individual strut includes a minimum length and a maximum length. Intraoperatively, surgeons may mount the first fixation component to the patient. Next, the surgeon may mount the second fixation component to the patient. Finally, the surgeon may interconnect the two components with the adjustable struts.

Despite the clinical success of such correction devices in orthopaedic applications, a number of challenges remain. For example, intraoperatively, surgeons must carefully plan the application and position of the first and second fixation components because the defined strut ranges (e.g., maximum and/or minimum length of each strut) limit how close and how far apart the first and second fixation components may be mounted from each other. If not properly planned, the surgeon may be unable to interconnect the first and second fixation components and may have to repeat the mounting process. Additionally, and/or alternatively, the struts may need to be changed out postoperatively with either longer or shorter struts during treatment.

Additionally, orthopaedic deformity correction devices, such as the Taylor Spatial Frame, may utilize software packages (typically web-based) to virtually align bone segments and to assist with the generation of the prescription. For clarification, as will be described in greater detail below, the software for generating the prescription will be referred to as the “prescription software” throughout this document. The prescription may be or may specify a strut adjustment schedule for an installed correction device. These prescription software packages, applications, components, or modules (used interchangeably herein without the intent to limit) require surgeons to input multiple parameters to fully process a surgical case. Some of the prescription software inputs, such as deformity parameters, may be obtained postoperatively from medical imaging. However, other prescription software inputs, such as the lengths of each strut, must be gathered from the correction device which is attached to the patient during surgery.

In use, correction devices are typically designed so that the intraoperative surgical procedure of attaching the hardware and the prescription software are completely separate. Surgeons typically install the hardware on the patient and then use the software postoperatively. Some software applications allow/require some preoperative planning within the software and then final adjustments may be made in the software postoperatively. In either scenario however, the separation of hardware and software means that it is easy for a surgeon to forget to record all of the necessary inputs for the prescription software during installation. If required software inputs are not gathered during surgery and cannot be obtained from medical images, a follow-up visit with the patient may be required to obtain the missing information.

Additionally, aside from the required prescription software inputs, surgeons often record a variety of notes during a case. These notes must be inputted into the software postoperatively if the surgeon wishes to have the notes for the case available within the prescription software.

Postoperatively, management of a patient remains a challenge as well. Generally speaking, patients are given a prescription (e.g., a prescribed strut adjustment schedule) defining specific strut adjustments to achieve the final, desired bone position, and are responsible for following the prescription. Depending on the location and orientation of the correction device, visualization of the adjustment scale located on each strut and/or making the required adjustments may be a difficult task for the patient to handle alone. That is, postoperatively, during the adjustment phase of treatment, the struts must be lengthened or shortened according to the prescription. As such, postoperatively, the success of a correction device is largely dependent on the patient. The patient must follow the prescription of strut adjustments correctly in order to achieve a good result. However, as stated above, visualization of a strut length via the physical scales on their frames may be difficult depending on how and where the struts are mounted. Additionally, if a maximum or minimum length of a strut is reached, the strut must be removed and replaced by a different sized strut if additional lengthening or shortening is required so that additional adjustments may be made.

Accordingly, it would be advantageous to provide an improved system including devices and methods for tracking the position of the fixation components, intraoperatively and postoperatively, and which may link the intraoperative surgical procedure and the postoperative prescription software into a seamless, integrated software system. It is with these considerations that the present disclosure is put forth.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

The present disclosure provides an intraoperative external fixation component tracking system to enable a surgeon to efficiently plan out construction of an external fixator. The intraoperative external fixation component tracking system further enables data related to the surgery, including data for determining a strut adjustment schedule for the external fixator, to be captured intraoperatively for use postoperatively. The present disclosure further provides a postoperative external fixation component tracking system to enable a patient to efficiently adjust struts of an installed external fixator. The postoperative external fixation component tracking system further enables to surgeon to remotely monitor the patient's compliance with the strut adjustment schedule.

In one embodiment, an electronic device is disclosed. The electronic device may include a storage device, a display, and a controller. The controller may be coupled to the storage device and the display. The controller may be configured to receive one or more inputs for determining a strut adjustment schedule for a patient during a surgical procedure for installing an external fixator on the patient, receive additional data related to the surgical procedure during the surgical procedure, store the one or more inputs for determining the strut adjustment schedule and the additional data in the storage device organized by a patient identification associated with the patient and by a procedure identification associated with the surgical procedure, display the one or more inputs for determining the strut adjustment schedule and the additional data on the display, and transmit, automatically, the one or more inputs for determining the strut adjustment schedule and the additional data to a remote device after completion of the surgical procedure.

In one embodiment, the one or more inputs for determining the strut adjustment schedule may include a size of each external fixation component of the external fixator.

In one embodiment, the one or more inputs for determining the strut adjustment schedule may include a type of each external fixation component of the external fixator.

In one embodiment, the one or more inputs for determining the strut adjustment schedule may include a mounting location of each external fixation component of the external fixator.

In one embodiment, the one or more inputs for determining the strut adjustment schedule may include a type of each strut attached to each external fixation component of the external fixator.

In one embodiment, the one or more inputs for determining the strut adjustment schedule includes a length of each strut.

In one embodiment, the length of each strut is received through user manipulation of a user interface of the electronic device.

In one embodiment, the length of each strut is received automatically from a tracking system attached to the external fixator.

In one embodiment, the length of each strut is received wirelessly from the tracking system attached to the external fixator.

In one embodiment, the additional data related to the surgical procedure includes at least one of textual data and visual data.

In one embodiment, a tracking system is disclosed. The tracking system may include a first tracking system component configured to be coupled to a first external fixation component of an external fixator and a second tracking system component configured to be coupled to a second external fixation component of the external fixator. The first tracking system component may include a controller. The controller may determine, in real-time, position data indicating a relative position between the first and second external fixation components based on data from the first tracking system component. The controller may wirelessly transmit the determined position data to a remote device.

In one embodiment, the first tracking system component may include an optical sensor.

In one embodiment, the optical sensor is an optical camera.

In one embodiment, the second tracking system component is an LED target.

In one embodiment, the controller wirelessly transmits the determined position data to the remote device during a surgical procedure for installing the external fixator on a patient.

In one embodiment, the determined position data is used to determine the mounting position for the second external fixation component relative to the known mounting position of the first external fixation component during the surgical procedure.

In one embodiment, the determined position data is used to determine a length and a type of each strut to attach to the first and second external fixation components during the surgical procedure.

In one embodiment, the controller determines a length of each strut attached to the first and second external fixation components based on the determined position data after completion of the surgical procedure.

In one embodiment, the controller wirelessly transmits the determined length of each strut to the remote device after completion of the surgical procedure.

In one embodiment, the determined length of each strut is used to verify compliance with a strut adjustment schedule associated with the external fixator.

Embodiments of the present disclosure provide numerous advantages. For example, during surgery, the intraoperative external fixation component tracking system enables the surgeon to efficiently plan out construction of an external fixator, thereby ensuring minimal strut change outs. The intraoperative external fixation component tracking system also enables data related to the surgery, including data for determining a strut adjustment schedule for the external fixator, to be captured intraoperatively for use postoperatively. Additionally, the postoperative external fixation component tracking system enables a patient to efficiently adjust struts of an installed external fixator. The postoperative external fixation component tracking system further enables the surgeon to remotely monitor the patient's compliance with the strut adjustment schedule and provides a feedback loop between the surgeon and patient to ensure proper care of the patient.

Further features and advantages of at least some of the embodiments of the present invention, as well as the structure and operation of various embodiments of the present invention, are described in detail below with reference to the accompanying drawings.

### BRIEF DESCRIPTION OF THE DRAWINGS

By way of example, a specific embodiment of the disclosed device will now be described, with reference to the accompanying drawings, in which:

FIG. 1 illustrates an embodiment of an intraoperative external fixation component tracking system in accordance with the present disclosure;

FIG. 2 illustrates an embodiment of an external fixator and a tracking system depicted in FIG. 1;

FIG. 3 illustrates an embodiment of a postoperative external fixation component tracking system in accordance with the present disclosure;

FIG. 4 illustrates an embodiment of a user interface provided by a patient computing device depicted in FIG. 3;

FIG. 5 illustrates a block diagram of an embodiment of a computing device in accordance with the present disclosure; and

FIG. 6 illustrates a block diagram of an embodiment of the tracking system depicted in FIGS. 1 and 3.

The drawings are not necessarily to scale. The drawings are merely representations, not intended to portray specific parameters of the disclosure. The drawings are intended to depict example embodiments of the disclosure, and therefore are not be considered as limiting in scope. In the drawings, like numbering represents like elements.

### DETAILED DESCRIPTION

For the purposes of promoting an understanding of the principles of the present disclosure, reference will now be made to example embodiments. It will nevertheless be understood that no limitation of the scope of the disclosure is thereby intended. Any alterations and further modifications in the described embodiments, and any further applications of the principles of the present disclosure as described herein are contemplated as would normally occur to one skilled in the art to which the disclosure relates.

The present disclosure is directed to a system and method for monitoring and/or tracking the relative position of external fixation components such as, for example, first and second external fixation frames or rings, both intraoperatively and postoperatively. Relative position data may be transmitted from the tracking system to a software system (including one or more software applications). The position data may be used, intraoperatively, by the surgeon to aid in properly mounting the external fixation components (e.g., first and second fixation frames or rings) to the patient thus eliminating the need for surgeons to pre-build their frames and/or to experiment with mounting locations during the surgical procedure. Postoperatively, the software may monitor the relative position data measured by the tracking system to provide a patient-surgeon feedback loop. Additionally, the position data may also be made available/visible to patients to aid in achieving the prescription specifying strut adjustments to be made over time.

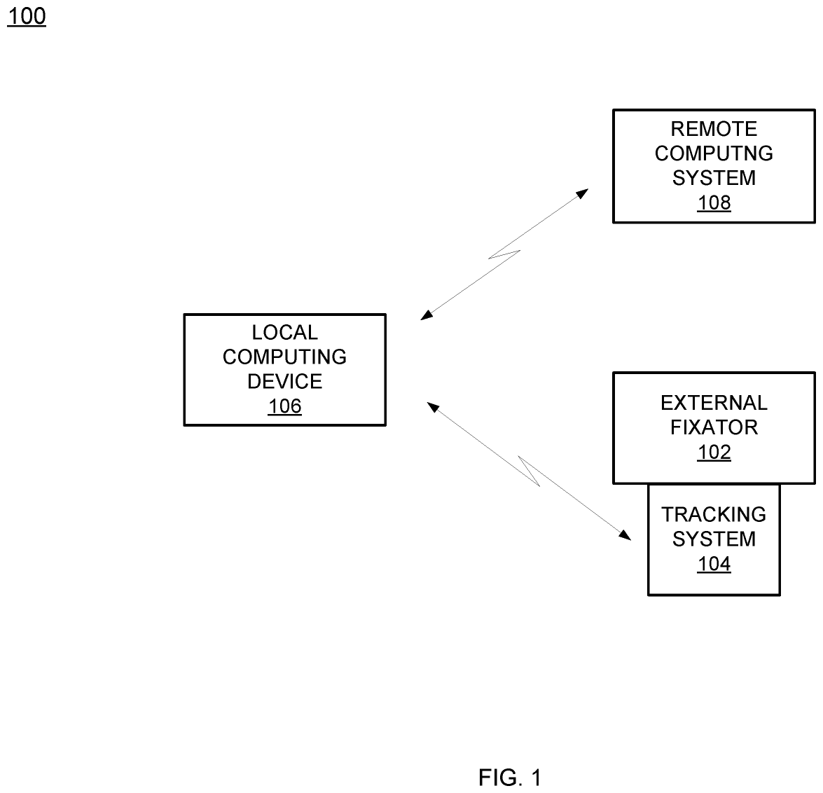

FIG. 1 illustrates an embodiment of an intraoperative external fixation component tracking system 100. The intraoperative external fixation component tracking system 100 may be used to track the relative positions of external fixation components of an external fixator during an installation procedure for the external fixator. As a result, a surgeon may install the external fixator more efficiently without a need to pre-build the external fixator and/or to experiment with mounting locations for the external fixation components. Additionally, the surgeon may install the external fixator with confidence that a strut adjustment schedule (e.g., a prescription) for the external fixator may be realized with minimal change outs of the initial struts. Further, the intraoperative external fixation component tracking system 100 enables any information related to the patient, installed external fixator, or installation procedure to be collected intraoperatively for use postoperatively, including for generation of the strut adjustment schedule.

As shown in FIG. 1, the intraoperative external fixation component tracking system 100 may include an external fixator 102, a tracking system 104, a local computing device 106, and a remote computing system 108. The external fixator 102 may be any bone alignment device or correction device now known or hereafter developed. The external fixator 102 may include first and second fixation frames or rings connected by one or more struts. The tracking system 104 may be any tracking system now known or hereafter developed. The tracking system 104 may be coupled to the external fixator 102 and may track the relative positions of the first and second fixation frames or rings of the external fixator 102.

The local computing device 106 may be any suitable computing device now known or hereafter developed including, for example, a smartphone, a tablet, a laptop, a notebook, a netbook, a personal computer (PC), etc. The remote computing system 108 may be any suitable remote computing system now known or hereafter developing including, for example, a remote computing device, a remote computer network, or a remote cloud network or platform.

The tracking system 104 may communicate directly or indirectly with the local computing device 106 and/or the remote computing system 108 over any known wireless communication standard or protocol. The local computing device 106 may also communicate directly or indirectly with the remote computing system 108 over any known wireless communication standard or protocol. Example wireless connections and/or protocols may include, for example, Wi-Fi (e.g., any IEEE 802.11 a/b/g/n network), Bluetooth, Bluetooth Low Energy (BLE), Near-Field Communication (NFC), any cellular communication standard, any infrared communication protocol, etc.

In various embodiments, the relative positions of the first and second fixation frames or rings of the external fixator 102 may be detected by the tracking system 104 and reported to the local computing device 106. The local computing device 106 may be consulted during installation of the external fixator 102 to plan and properly position the first and second fixation frames or rings of the external fixator 102. The local computing device 106 may transfer information regarding the installation of the external fixator 102 provided by the tracking system 104 to the remote computing system 108 for postoperative use as described herein.

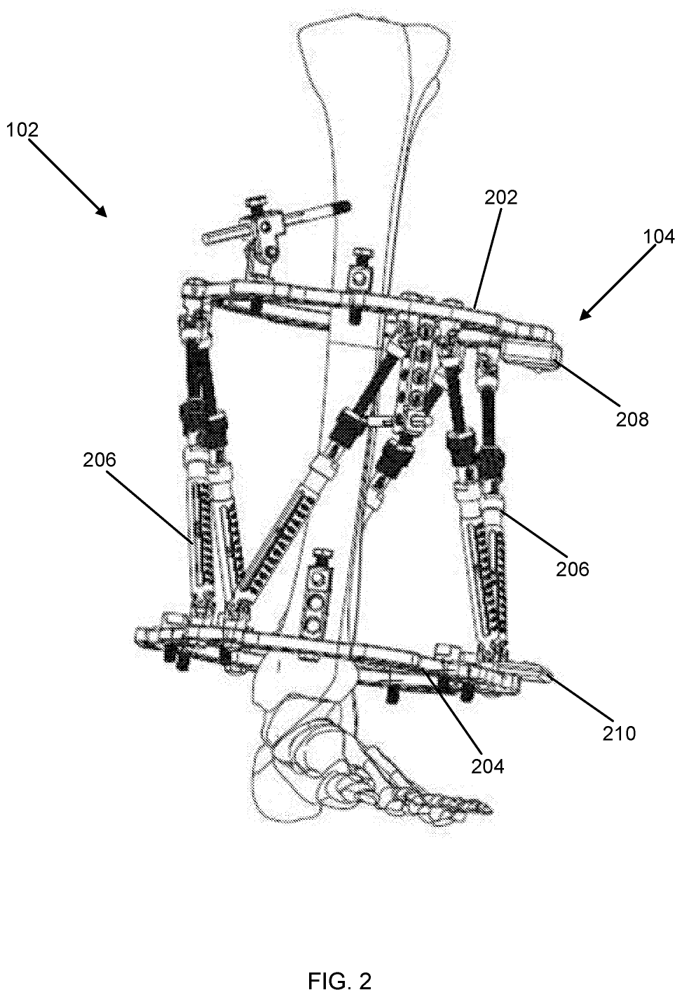

FIG. 2 illustrates an embodiment of the external fixator 102 and the tracking system 104 depicted in FIG. 1. The external fixator 102 may include a first external fixation component 202 (e.g., a first fixation frame or ring) and a second external fixation component 204 (e.g., a second fixation frame or ring). The first and second external fixation components 202 and 204 may be connected by one or more struts 206. Six struts 206 are shown connecting the first and second external fixation components 202 and 204 but the external fixator 102 is not so limited. That is, any number of struts 206 may connect the first and second external fixation components 202 and 204.

The tracking system 104 may include a first tracking system component 208 and a second tracking system component 210. The first tracking system component 208 may be connected to the first external fixation component 202 while the second tracking system component 210 may be connected to the second external fixation component 204.

In one embodiment, the first tracking system component 208 may be or include an optical sensor such as, for example, an optical camera and the second tracking component 210 may be a target such as, for example, an LED target. In an alternative embodiment, the first tracking system component 208 may be a non-optical sensor. In general, the first and second tracking system components 208 and 210 may be components of any sensor system now known or hereafter developed that may monitor and/or track relative positions of the first and second tracking system components 208 and 210 and, consequently (e.g., indirectly), relative positions of the first and second external fixation components 202 and 204. In one embodiment, the first tracking system component 208 may be or include a laser-based sensor or an infrared-based sensor.

In use, the first tracking system component 208 (e.g., optical camera) tracks the relative position of the second tracking system component 210 (e.g., target) in space to provide relative positional data in all six degrees of freedom in real-time (e.g., corresponding to the six struts 206). It should be understood that while the present disclosure will be described and illustrated in terms of fixation frames or rings, it is envisioned that the tracking system 104 may be used in connection with other external fixation components such as, for example, a linear bone transport frame. The first tracking system component 208 may determine a distance between the first and second external fixation components 202 and 204 in real-time. The distance may be positional data of the first and second external fixation components 202 and 204 and may be based on any data, signal, or information provided by the sensor of the first tracking system component 208. The first tracking system component 208 may also determine a length of each strut 206. The length of each strut 206 may be based on the determined distance between the first and second external fixation components 202 and 204.

The tracking system 104 may include a transceiver to facilitate wireless communications with the local computing device 102 and/or the remote computing system 108. Alternatively, the tracking system 104 may be operatively coupled to any external computing device (e.g., the local computing device 102) via a hardwire connection.

In various embodiments, the tracking system 104 may be a small, lightweight, precise, and inexpensive optical tracking system. By mounting the tracking system 104 to the the external fixator 102, relative positional data of the first and second external fixation components 202 and 204 may be tracked and monitored. Further, the relative positional data of the first and second external fixation components 202 and 204 may be used to determine a length of each strut 206. The relative positional data of the first and second external fixation components 202 and 204 and the length data for each strut 206 may be provided to a user (e.g., a surgeon) through the local computing device 106 (e.g., provided on a display of the local computing device 106). The relative positional data of the first and second external fixation components 202 and 204 and the length data for each strut 206 may be provided to the local computing device 106 in real-time and automatically. In turn, any displayed relative positional data of the first and second external fixation components 202 and 204 and any displayed length data for each strut 206 on the local computing device 106 may be updated dynamically as the first and second external fixation components 202 and 204 are moved relative to one another or if any strut 206 is adjusted.

Intraoperatively, a surgeon may initially mount the first external fixation component 202 to a patient with the first tracking system component 208 mounted thereon. Next, the surgeon may position the second external fixation component 204 on the patient with the second tracking system component 210 mounted thereon. Utilizing software associated with the intraoperative external fixation component tracking system 100, the surgeon may properly position the second external fixation component 204 relative to the first mounted external fixation component 202 before fully installing the second external fixation components 204 to the patient. In general, the first and second external fixation components 202 and 204 may be mounted to the patient in any order.

In various embodiments, the tracking system 104 and associated software may enable the surgeon to select component parameters (e.g., fixation component type, fixation component size, etc.) and candidate initial positions for the first and second external fixation components 202 and 204 that ensure the first and second external fixation components 202 and 204 may be connected by available struts 206. Further, the tracking system 104 and associated software may estimate a strut adjustment schedule for the patient based on the candidate initial positions of the first and second external fixation components 202 and 204 and other necessary software inputs. Based on the candidate initial positions and the estimated strut adjustment schedule, the tracking system 104 and associated software may predict a likelihood that any of the struts 206 may need to be changed out (e.g., for shorter or longer struts over the length of time the patient wears the external fixator 102 in accordance with the estimated strut adjustment schedule). This allows the surgeon to intelligently select the initial mounting positions for the first and second external fixation components 202 and 204, as well as the initial struts 206, in a manner that ensures constructability with minimal change out of any strut 206.

As previously mentioned, the intraoperative external fixation component tracking system 100 may include or be associated with a software system including one or more software applications. The software applications may be provided by the local computing device 106, the remote computing system 108, or the tracking system 104, either individually or collectively. The software applications may be provided by a remote server and may be web-based or may reside on the local computing device 106, the remote computing system 108, and/or the tracking system 104. In one embodiment, the software system may include an intraoperative software application, a prescription software application (or correction analysis application), and a patient software application, each of which is described further herein.

In an embodiment, the intraoperative software application may be provided by the local computing device 106 (e.g., through a web-based server). The intraoperative software application may be used by a sales representative or surgical staff to collect and organize data related to a surgical procedure in real-time during a surgical procedure. For example, the interactive software application may allow notes, photos, videos, and other surgical parameters to be gathered and organized during the surgical procedure. The collected data may then be provided to the remote computer system 108 for storage and further use as described herein.

In one embodiment, the intraoperative software may be associated with the tracking system 104 mounted on the external fixator 102 and may be used intraoperatively to assist with a procedure for mounting the external fixator 102 on the patient as described herein. That is, for example, the tracking system 104 and intraoperative software may be used intraoperatively to display data (e.g., positional data for the first and second external fixation components 202 and 204 and/or length data for any strut 206) in real-time on the local computing device 106. The intraoperative software may therefore enable the surgeon to efficiently plan the initial mounting positions of the first and second external fixation components 202 and 204 as described herein.

For example, intraoperatively, the relative positional data between the first and second tracking system components 208 and 210 may be transmitted to the intraoperative software residing on the local computing device 106. Using the intraoperative software, the surgeon may adjust the relative positions of the first and second external fixation components 202 and 204 until the surgeon is satisfied that the lengths of the struts 206 are within the physical constraints of the external fixator 102, ensuring that the external fixator 102 is buildable. Since the lengths of the struts 206 may be made visible through the intraoperative software, the surgeon may also manipulate the positions of the first and second external fixation components 202 and 204 to avoid change outs for the struts 206 (e.g., replacing existing struts with longer or shorter struts) early on in the prescription. With some preoperative planning including patient's deformity parameters and the reference ring position, the tracking system 104 may communicate with the intraoperative software to actively solve the final strut adjustment lengths during application of the external fixator 102.

That is, in one embodiment, the relative positions of the first and second tracking system components 208 and 210 may be used to define the lengths of the hardware components (e.g., struts 206) connecting the first and second external fixation components 202 and 204 together. As the first and second external fixation components 202 and 204 are manipulated into position, the surgeon may view the lengths of the struts 206 that will be required for the external fixator 102 being constructed. As a result, the surgeon may avoid building an external fixator 102 outside of the physical constraints of the struts 206 and may optimize the position of the first and second external fixation components 202 and 204 before attaching them to the patient.

Additionally, the intraoperative software providing an active final solution may assist surgeons to position the first and second external fixation components 202 and 204 in an orientation that may optimize or eliminate change out of the struts 206 over the course of an entire prescription. As a result, the intraoperative external fixation component tracking system 100 may improve the surgeon's confidence during application of the external fixator 102 and may minimize the amount of time spent changing out the struts 206 in clinic.

Additionally, in use, the intraoperative software could be used by the surgeon for preoperative planning of the deformity and identifying the preferred mounting locations of the first and second external fixation components 202 and 204. For example, in one embodiment, if so desired, using the intraoperative software, the surgeon could elect to preoperatively plan or calculate the postoperative prescription for correcting the deformity during application of the external fixator 102. Thereafter, intraoperatively, the desired prescription for the lengths of the struts 206 may be adjusted in real-time as the surgeon manipulates the position of the first and second external fixation components 202 and 204. This method allows the surgeon to minimize or potentially eliminate change outs of the struts 206 throughout the postoperative prescription.

In an embodiment, the intraoperative software provided by the local computing device 106 may provide the surgeon with the preoperative and postoperative planning described herein based on monitoring and/or tracking data provided by the tracking system 104.

Once the positions of the first and second external fixation components 202 and 204 are fixed, the lengths of the struts 206 of the constructed external fixator 102 may be transmitted or made accessible to a surgeon through a surgeon facing software or portal thereby eliminating the need to manually input the values into the prescription generating software. As a result, utilization of the tracking system 104 and associated software according to the present disclosure enables the inputs for the prescription software application to be more precise. Additionally, at the conclusion of the surgical procedure, the surgeon could complete any remaining steps required to create or finalize the strut adjustment prescription for the patient.

Additionally, the intraoperative software may allow surgeons to record additional organized case parameters, notes, and photos during surgery. That is, intraoperatively, in one embodiment, the intraoperative software allows surgeons to record case parameters during surgery on the local computing device 106. For example, the intraoperative software could allow any data relating to the patient, the procedure, or the constructed external fixator 102 to be recorded including for example, the sizes and/or types of the first and second external fixator components 202 and 204, the lengths of the struts 206, the type of struts 206, the mounting locations of the struts 206 and/or the first and second external fixator components 202 and 204, and any other parameters that may be used to generate a patient's strut adjustment prescription, etc.

Additionally, the intraoperative software could also facilitate storage of photos, notes, etc. taken during the surgery, which could also be organized by case and/or patient. For example, pictures could provide valuable information about the patient's soft tissue and construction of the eternal fixator 102 that might not be easily discernible from medical images. In general, the intraoperative software may be used to capture intraoperatively any data or information that may be used to postoperatively generate a strut adjustment schedule as well as capture any other data that may be related to the procedure for installing the external fixator 102, with at least some of the data or information being provided to the intraoperative software in real-time and/or automatically from the tracking system 104. Further, the intraoperative software may provide any of the capture data to the prescription software automatically such that inputs for determining the strut adjustment schedule are pre-populated based on the provided data.

In various embodiments, the intraoperative software may be interactive software that automatically loads and/or displays captured inputs that a user may view and manipulate. In various embodiments, the intraoperative software may present captured inputs in one or more pre-populated fields and may provide an interactive PDF file or form. The intraoperative software may provide a visual rendering (e.g., CAD rendering) of the external fixator 102 as it is being constructed.

The prescription software application may be provided by the local computing device 106 or the remote computing system 108. In an embodiment, the prescription software application (or correction analysis software) is provided by the remote computing system 208. The prescription software application may generate a strut adjustment schedule based on information provided by the intraoperative software application provided by the local computing device 106 or by direct input. In an embodiment, after completion of the surgery, the data from the intraoperative software could be uploaded to the prescription software (e.g., manually or automatically), which could be used by the surgeon to generate the patient's prescription. The data uploaded to the prescription software application may be organized by case and/or patient. For example, the remote computing system 108 may include a database for storing case parameters for one or more patients organized by the patient and/or procedure.

Since the data is organized according to the case/patient, the surgeon may easily generate a new case for the patient on the prescription software with any inputs from the intraoperative software pre-populated.

The prescription software may be accessible to any computing device communicatively coupled to the remote computing system 108. After completion of the installation procedure for the external fixator 102, the surgeon may complete any remaining steps of the pre-populated prescription software program to create the strut adjustment schedule or prescription for the patient. The strut adjustment schedule may be stored by the remote computing system 108 and may be made accessible to other computing devices as described further herein.

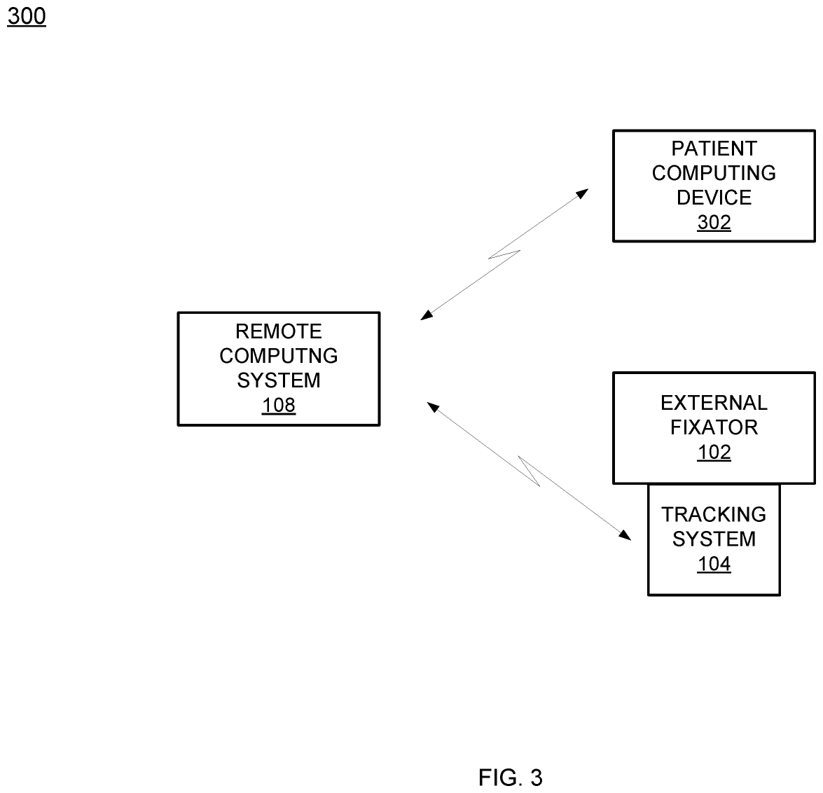

FIG. 3 illustrates an embodiment of a postoperative external fixation component tracking system 300. The postoperative external fixation component tracking system 300 may be used to track the relative positions of external fixation components of an external fixator after an installation procedure for the external fixator. As a result, a surgeon may monitor a patient's compliance with a strut adjustment schedule and may provide modifications to the strut adjustment schedule to the patient. Additionally, any information from the patient including, for example, any notes, photos, or reports, may be provided to the surgeon to enable a surgeon-patient feedback system that improves the experience of the patient and increases the likelihood of success of the treatment of the patient. As will be described in greater detail herein, the postoperative external fixation component tracking system 300 includes a tracking system. In use, the tracking system for the postoperative external fixation component tracking system 300 may be the same or substantially similar to the tracking system 104 used in the intraoperative external fixation component tracking system 100. However, as will be appreciated by one of ordinary skill in the art, the intraoperative and the postoperative external fixation component tracking systems 100, 300 may include different components (though they may also share many of the same components).

As shown in FIG. 3, the postoperative external fixation component tracking system 300 may include the external fixator 102, the tracking system 104, a patient computing device 302, and the remote computing system 108. The patient computing device 302 may be any suitable computing device now known or hereafter developed including, for example, a smartphone, a tablet, a laptop, a notebook, a netbook, a personal computer (PC), etc.

The tracking system 104 may communicate directly or indirectly with the patient computing device 302 over any known wireless communication standard or protocol. The patient computing device 302 may also communicate directly or indirectly with the remote computing system 108 over any known wireless communication standard or protocol. Example wireless connections and/or protocols may include, for example, Wi-Fi (e.g., any IEEE 802.11 a/b/g/n network), Bluetooth, Bluetooth Low Energy (BLE), Near-Field Communication (NFC), any cellular communication standard, any infrared communication protocol, etc.

In an embodiment, the remote computing system 108 may provide the prescription software that determines a strut adjustment schedule for the patient based on the installed external fixator 102. Further, the patient computing device 302 may provide the patient software application. The strut adjustment schedule generated for the patient by the remote computing system 108 may be provided to the patient software application provided by the patient computing device 108. The strut adjustment schedule may specify adjustments to be made to each strut 206 of the external fixator 102 over a period of time the patient is expected to wear the external fixator 102.

In an embodiment, the patient software application may present the strut adjustment schedule to the patient on a display of the patient computing device 302. Any modifications to an original strut adjustment schedule may be provided to the patient software application from the prescription software and may also be presented to any user of the patient computing device 302. Additionally, any notifications related to the strut adjustment schedule or any reminders to adjust the struts 206 may be provided to the patient software application from the prescription software. Alternatively, reminders to adjust the struts 206 may be provided by the patient software application directly based on a stored strut adjustment schedule.

In an embodiment, adjustments to the struts 206 may be detected by the tracking system 104 and provided to the patient software application provided by the patient computing device 302. The patient computing device 302 may upload any detected adjustments to the struts 206 to the prescription software provided by the remote computing system 208. The surgeon, a medical caregiver, or any other authorized individual may be provided with the detected adjustments of the struts 206 through the remote computing system 108 either directly or through use of any authorized computing device communicatively coupled to the remote computing system 108. In this manner, the surgeon, medical caregiver, or other authorized individual may monitor and track the patient's compliance with the strut adjustment schedule. Additionally, any information uploaded to the remote computing system 108 by the patient may be reviewed to determine an overall progress or health of the patient with regard to the external fixator 102.

The postoperative external fixation component tracking system 300 enables, postoperatively, lengths of the struts 206 to be displayed to the patient on the patient computing device 302 through the patient software application, thereby making length values of the struts 206 easier to see and comprehend. For example, in one embodiment, displaying the length of each strut 206 on the patient computing device 302 enables easier and better resolution of the current position of the struts 206 than might be obtained through any other length determination mechanism provided by the struts 206. That is, depending on the type of the external fixator 102, it may be difficult for the patient to see the physical scale located on each strut 206 when making an adjustment. As a result, it may be difficult for the patient to accurately adjust the relative position of the struts 206 according to the required prescription. Utilizing the tracking system 104 and the patient software, however, the patient may adjust the length of the struts 206 while viewing a user-friendly display that enables easier determination of strut 206 adjustment to be made. Consequently, any accidental and/or incorrect adjustments of any strut 206 may be avoided.

Additionally, incorporation of the tracking system 104 with the external fixator 102 enables a surgeon-patient feedback loop, which is not dependent on patient compliance as with any software system that requires the patient to manually record adjustments to the struts 206. That is, in one embodiment, the tracking system 104 provides a surgeon-patient feedback loop during the adjustment phase of treatment without requiring additional patient input. As an example, the postoperative external fixation component tracking system 300 may actively monitor the position of the first and second external fixation components 202 and 204 and/or lengths of the struts 206 and may compare such information to the patient's prescription. The position of the first and second external fixation components 202 and 204 and/or lengths of the struts 206 may be provided directly to the remote computing system 108 from the tracking system 104 or may be provided indirectly through the patient computing device 302.

The detected positional data and/or length data may then be monitored by the surgeon or other authorized individual through the remote computing system 108. Accordingly, the postoperative external fixation component tracking system 300 may actively monitor the position of the first and second external fixation components 202 and 204 and/or lengths of the struts 206 directly, thus eliminating the need for patient input or any associated incorrect reporting of the lengths of the struts 206. In one embodiment, the surgeon and patient could be immediately notified through the software system described herein if the struts 206 are being adjusted in a way that does or does not match the prescription.

Thus, at the conclusion of the surgical procedure installing the external fixator 102, the patient may return home with the required prescription and instructions to adjust the struts 206 according to the prescription. The tracking system 104 may remain coupled to the first and second external fixation components 202 and 204 with the patient postoperatively to monitor progress. The patient software and/or the patient computing device 302 may store an electronic copy of the patient's prescription that may be used to display updated lengths of the struts 206 as the patient adjusts the struts 206. The patient software may also provide feedback to the surgeon through the prescription software and remote computing system 108 so that the surgeon may monitor the status of the patient's external fixator 102 postoperatively. Surgeons and patients may be immediately notified if adjustments to the struts 206 are not following the prescription. As a result, the postoperative external fixation component tracking system 300 provides patients and surgeons greater confidence between clinical visits while simplifying the adjustments process.

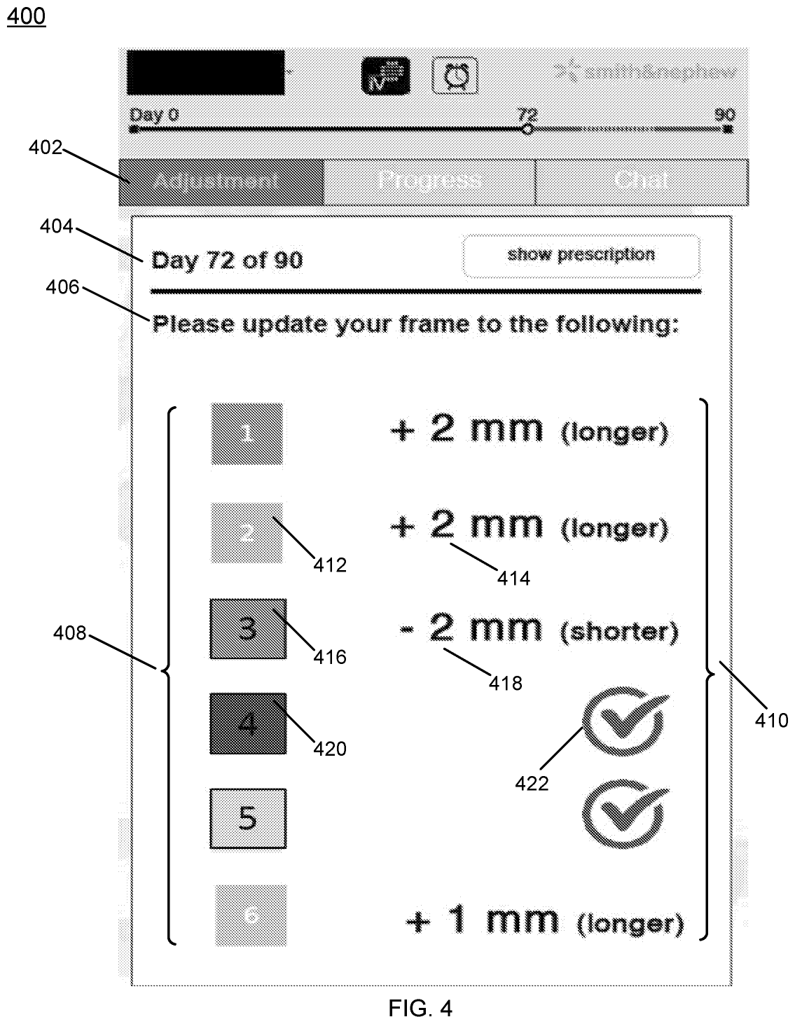

FIG. 4 illustrates an embodiment of a user interface 400 provided by the patient computing device 302. The user interface 400 may be a portion of a display provided by the patient software. In one embodiment, the user interface 400 may be provided the patient software as a mobile application (app). The user interface 400 may provide the patient with information and/or directions for adjusting a length of one or more of the struts 206 of the external fixator 102 in a clear and concise manner to improve the process for length adjustments to the struts 206, thereby improving a likelihood the patient complies with a prescribed strut adjustment schedule.

As shown in FIG. 4, the user interface 400 may include a first indicator 402 indicating that the user interface 400 specifies length adjustments to be made to the struts 206. The user interface 400 may also include a second indicator 404 indicating a timing for the adjustments (e.g., for which day the displayed adjustments are to be made). The user interface may further include a third indicator 406 indicating that certain length adjustments of the struts 206 are to be made. The first, second, and third indicators 402, 404, and 406 may include any combination of textual and/or graphical components as shown.

The user interface 400 may include icons or indicators 408 indicating each individual strut 206 of the external fixator 102 along with corresponding instructions 410 for adjusting the length of each strut 410 (if necessary). For example, a first strut indicator 412 is associated with a first corresponding instruction 414 specifying that the second strut 206 is to be extended by 2 millimeters (mm). A second strut indictor 416 is associated with a second corresponding instruction 418 specifying that the third strut 206 is to be shortened by 2 mm. A third strut indicator 420 is associated with an indicator 422 specifying that the fourth strut 206 is already at the correct length. The first, second, and third strut indicators 412, 416, and 420 may include any combination or numerical and graphical components as shown. The instructions 414 and 418 may include textual descriptions. The indicator 422 may be any graphical icon indicating a correct length of a strut 206.

The indicators 408 and corresponding instructions 410 may be generated by the patient software based on real-time information provided by the tracking system 104 and based on information provided by the prescription software from the remote computing system 108. One a strut 206 is properly adjusted in accordance with a provided instruction 410, any textual instruction may be dynamically updated to the icon 422 to quickly and efficiently convey to the patient that a particular strut 206 has been adjusted correctly.

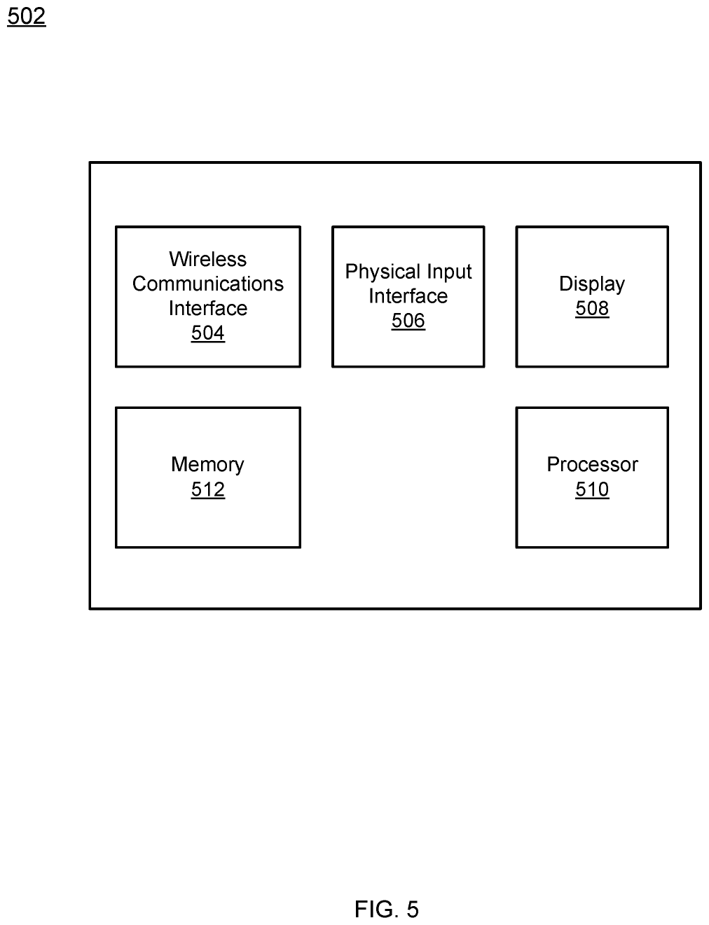

FIG. 5 illustrates an embodiment of a computing device 502. The computing device 502 may represent an implementation of the local computing device 106 or the patient computing device 302. Accordingly, FIG. 5 provides a block diagram of exemplary functional components of the local computing device 106 and/or the patient computing device 302.

The computing device 502 may include a wireless communications interface 504. The wireless communications interface 504 may provide interfaces for communicating with any local or remote device or network through any wireless communication technology.

The computing device 502 may include a physical input interface 506 for interfacing with one or more physical inputs that may be manipulated by a user. The physical input interface 506 may include or may be coupled to a variety of inputs including a keyboard, a mouse, a button, a knob, or any other type of user input feature or component such as, for example, a touchscreen. The physical input interface 506 may provide a way for a user to provide inputs to the computing device 502.

The computing device 502 may include a display 508. The display 508 may include a visual display that may render visual information and a display controller for controlling the rendering of any visual information. The visual information may be any graphical or textual information. The display 508 may include a touchscreen or a touch-sensitive display. Accordingly, the display 508 may provide visual information to a user and/or may receive input from the user.

The computing device 502 may further include a processor circuit or controller 510 and an associated memory component 512. The memory component 512 may store one or more programs for execution by the processor circuit 510 to implement one or more functions or features implemented by the local computing device 106 and/or the patient computing device 302 as described herein. The processor circuit 510 may be implemented using any processor or logic device. The memory component 512 may be implemented using any machine-readable or computer-readable media capable of storing data, including both volatile and non-volatile memory. Each component of the computing device 502 depicted in FIG. 5 may be coupled to the processor circuit 510 as well as any other depicted component. The depicted components may be implemented in hardware or software as appropriate, or any combination thereof

As previously mentioned, the computing device 502 may represent an implementation of the local computing device 106. As such, the computing device 502 may implement and/or provide any feature of the intraoperative software described herein. The computing device 502 may provide the intraoperative software as an app, as a portion of a web-based interface, or as an application residing on the computing device 502.

As providing the features and capabilities of the intraoperative software and/or the local computing device 106, the computing device 502 may provide one or more of the following: receive one or more inputs for determining a strut adjustment schedule for a patient during a surgical procedure for installing an external fixator on the patient; receive additional data related to the surgical procedure during the surgical procedure; store the one or more inputs for determining the strut adjustment schedule and the additional data in a memory storage device organized by a patient identification associated with the patient and by a procedure identification associated with the surgical procedure; display the one or more inputs for determining the strut adjustment schedule and the additional data on the display 508; and transmit the one or more inputs for determining the strut adjustment schedule and the additional data to a remote device after completion of the surgical procedure.

The one or more inputs for determining the strut adjustment schedule for the patient may include a size and/or a type each external fixation component of an external fixator (e.g., the external fixator 102) and/or a mounting position of each external fixation component of the external fixator. The one or more inputs for determining the strut adjustment schedule for the patient may further include a type and/or a length of each strut attached to the external fixation components of the external fixator. The length of each strut may be provided manually through user input (e.g., through a user interface provided by the physical input interface 506). The length of each strut may also be provided automatically, wirelessly, and/or in real-time from the tracking system 104. The additional data received by the computing device 502 include any type of textual data (e.g., notes) or visual data (e.g., photos or videos).

Any information received by the computing device 502 may be stored by the computing device 502 and/or transmitted to the remote computing device 108. The remote computing device 108 may use any information from computing device 502 to determine the strut adjustment schedule for the installed external fixator. The computing device 502 and/or the remote computing device 108 may store and/or organize any received information based a unique identifier for the patient (e.g., a patient identification) and/or a unique identifier for the surgical procedure (e.g., a surgical procedure identification).

The computing device 502 may use any received information regarding the positioning of the external fixation components of the external fixator to guide planning of installation or construction of the external fixator as described herein by, for example, displaying a visual representation of the planned external fixator on the display 508, displaying any calculated distances between the external fixation components, displaying any calculated strut lengths for a planned or constructed external fixator, and/or displaying any indications whether a planned or constructed external fixator may require a change out of struts.

As previously mentioned, the computing device 502 may represent an implementation of the patient computing device 302. As such, the computing device 502 may implement and/or provide any feature of the patient software described herein. The computing device 502 may provide the patient software as an app, as a portion of a web-based interface, or as an application residing on the computing device 502.

As providing the features and capabilities of the patient software and/or the patient computing device 302, the computing device 502 may receive real-time strut length data from the tracking system 104. The real-time strut length data may be provided on the display 508 for review by the patient. The computing device 502 may also provide the user interface 400 depicted in FIG. 4.

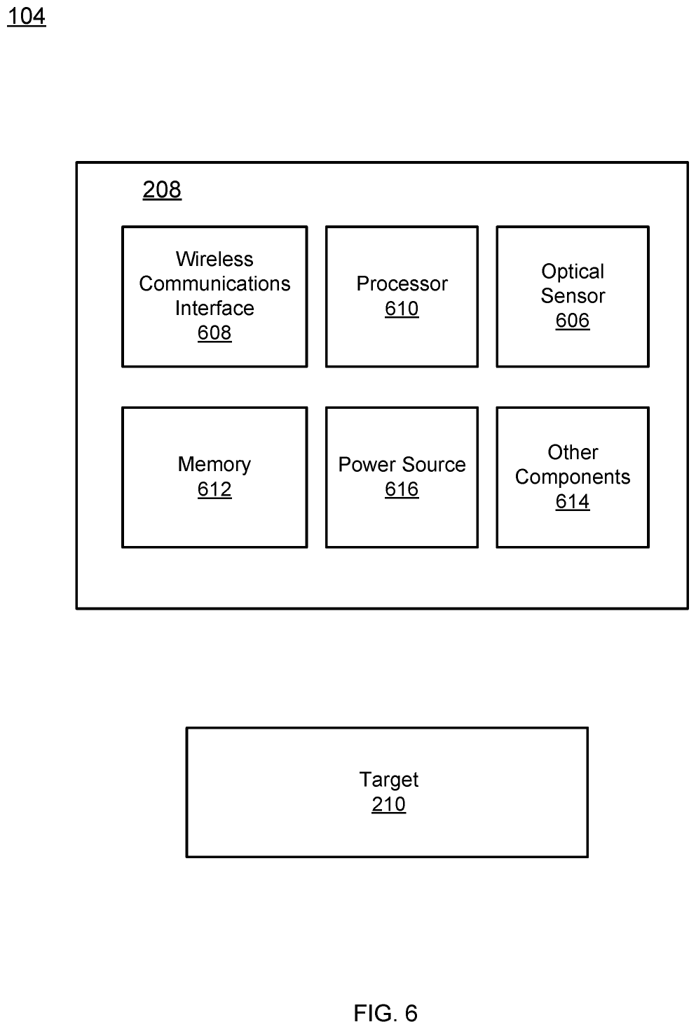

FIG. 6 illustrates a block diagram of exemplary functional components of the tracking system 104. As shown, the tracking system 104 may include the first tracking system component 208 and the second tracking system component 210. The first tracking system component 208 may be coupled to a first external fixation component of an external fixator (e.g., the external fixator 102). The second tracking system component 210 may be coupled to a second external fixation component of the external fixator. The first tracking system component 208 may include an optical sensor 606. The optical sensor 606 may be an optical camera. The second tracking system component 210 may be an LED target.

The first tracking system component 208 may include a wireless communications interface 608. The wireless communications interface 608 may provide interfaces for communicating with any local or remote device or network through any wireless communication technology.

The first tracking system component 208 may further include a processor circuit or controller 610 and an associated memory component 612. The memory component 612 may store one or more programs for execution by the processor circuit 610 to implement any functionality of the tracking system 104 as described herein. The processor circuit 610 may be implemented using any processor or logic device. The memory component 612 may be implemented using any machine-readable or computer-readable media capable of storing data, including both volatile and non-volatile memory. Each component of the first tracking system component 208 depicted in FIG. 6 may be coupled to the processor circuit 610 as well as any other depicted component. The depicted components may be implemented in hardware or software as appropriate, or any combination thereof

The first tracking system component 208 may also include other additional components 614. The additional components 614 may be any type of electrical and/or mechanical component. In various embodiments, the additional components 614 may include one or more additional sensors that may be incorporated into the tracking system 104 to provide one or more additional measurements or functions including the following types of sensors or functionality may be incorporated into the tracking system 104: temperature sensors; RFID tag or reader; accelerometer; pedometer, GPS receiver; moisture sensor; force sensors; pressure sensors; PH sensor; strain gauges and/or ability to receive strain gauge data; ultrasonic healing capability, etc. may be incorporated. Additionally, and/or alternatively, the tracking system 104 may include communication with a wearable device, such as, for example, a Fitbit or the like.

The first tracking system component 208 may also include a power source 616. The power source may be any suitable power source for powering any electronic component of the tracking system 104 such as, for example, an internal power source, an external power source, an inductive charging system, disposable batteries, rechargeable batteries, motion/inertial charging, etc.

In various embodiments, the tracking system 104 and/or any constituent component thereof may be mounted to the first and/or second external fixation components 202 and 204 (as appropriate) using any appropriate mechanism including, for example, fasteners, adhesive, welding, etc. In addition, the tracking system 104 and/or any constituent component thereof may be mounted to any other components of the tracking system 104. For example, in one embodiment, the optical sensor 606 may be mounted to the first external fixation component 208 and the corresponding target may be mounted to the second external fixation component 210. Alternatively, the target may be mounted to the first external fixation component 208 and the optical sensor 210 may be mounted to the second external fixation component 210. Alternatively, one or both of the optical system 606 and corresponding target may be mounted to one or more of the struts 206 or to another component of the external fixator 102. In various embodiments, the constituent components of the tracking system 104 may also act as fiducials for medical imaging.

The processor circuit 610 may operate to determine, in real-time, position data indicating a relative position between first and second external fixation components based on data from the first tracking system component 208. As an example, the first tracking system component 208 may generate data indicative of a distance between the first tracking system component 208 and the second first tracking system component 208. The data from the first tracking system component 208 may be provided to the processor circuit 610 which, in turn, may determine the position data of the first and second external fixation components. The processor circuit 610 may then wirelessly transmit the determined position data to a remote device using the wireless communications interface 608.

As described herein, the tracking system 104 may be used intraoperative and/or postoperatively. Accordingly, the position data may be transmitted to a first remote device (e.g., the local computing device 106) during a surgical procedure for installing an external fixator (e.g., to help guide or plan installation of the external fixator as described herein) and/or may be transmitted to a second remote device (e.g., the patient computing device 302) after completion of the surgical procedure (e.g., to verify compliance with a strut adjustment schedule associated with the external fixator).

During the surgical procedure, the determined position data may be used to determine a first mounting position for the first external fixation component and to determine a second mounting position for the second external fixation component during the surgical procedure. Further, the determined position data may be used to determine a length and a type of each strut to attach to the first and second external fixation components. The mounting positions and strut types and lengths may be used to plan and/or complete construction of the external fixator while minimizing any strut change outs.

After the surgical procedure, the controller may determine a length of each strut attached to the first and second external fixation components. The determined strut lengths may be provided to, for example, the patient computing device 302, to facilitate compliance with a strut adjustment schedule as described herein.

Note, while the various software applications (e.g., the intraoperative software application, the prescription software application, and the patient software application) are described as being separate software applications, it is envisioned that they could be fully integrated software systems that allow for easy transfer and/or access to data therebetween. Utilization of the intraoperative software applications to capture prescription software inputs significantly reduces the postoperative time needed for a surgeon to manually input the needed information for the prescription software.

In one embodiment, in use, the intraoperative software application could be linked to any system for measuring strut lengths or ring locations intraoperatively, and could include one or more of the following combinations of functionality: prescription software inputs, prescription software inputs and notes, prescription software inputs and photos, prescription software inputs and connectivity to sensor technology, prescription software inputs, notes, and photos, prescription software inputs, notes, photos, and connectivity to sensor technology, etc.

While the present disclosure refers to certain embodiments, numerous modifications, alterations, and changes to the described embodiments are possible without departing from the sphere and scope of the present disclosure, as defined in the appended claim(s). Accordingly, it is intended that the present disclosure not be limited to the described embodiments, but that it has the full scope defined by the language of the following claims, and equivalents thereof. The discussion of any embodiment is meant only to be explanatory and is not intended to suggest that the scope of the disclosure, including the claims, is limited to these embodiments. In other words, while illustrative embodiments of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more embodiments or configurations for the purpose of streamlining the disclosure. However, it should be understood that various features of the certain embodiments or configurations of the disclosure may be combined in alternate embodiments, or configurations. Moreover, the following claims are hereby incorporated into this Detailed Description by this reference, with each claim standing on its own as a separate embodiment of the present disclosure.

As used herein, an element or step recited in the singular and proceeded with the word “a” or “an” should be understood as not excluding plural elements or steps, unless such exclusion is explicitly recited. Furthermore, references to “one embodiment” of the present disclosure are not intended to be interpreted as excluding the existence of additional embodiments that also incorporate the recited features.

The phrases “at least one”, “one or more”, and “and/or”, as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

What is claimed:

1. An electronic device, comprising:
a storage device;
a display; and
a controller, the controller coupled to the storage device and the display, the controller to:
receive one or more inputs for determining a strut adjustment schedule for a patient during a surgical procedure for installing an external fixator on the patient;
receive additional data related to the surgical procedure during the surgical procedure;
store the one or more inputs for determining the strut adjustment schedule and the additional data in the storage device;
display the one or more inputs for determining the strut adjustment schedule and the additional data on the display; and
transmit electronically the one or more inputs for determining the strut adjustment schedule and the additional data to a remote device after completion of the surgical procedure.

2. The electronic device of claim 1, wherein the one or more inputs for determining the strut adjustment schedule and the additional data are stored organized by a patient identification associated with the patient.

3. The electronic device of claim 1, wherein the one or more inputs for determining the strut adjustment schedule and the additional data are stored organized by a procedure identification associated with the surgical procedure.

4. The electronic device of claim 1, wherein the one or more inputs for determining the strut adjustment schedule and the additional data are transmitted automatically.

5. The electronic device of claim 1, wherein the one or more inputs for determining the strut adjustment schedule and the additional data are transmitted after approval by a user.

6. The electronic device of claim 1, wherein the one or more inputs for determining the strut adjustment schedule includes a size of each external fixation component of the external fixator.

7. The electronic device of claim 6, wherein the one or more inputs for determining the strut adjustment schedule includes a type of each external fixation component of the external fixator.

8. The electronic device of claim 7, wherein the one or more inputs for determining the strut adjustment schedule includes a mounting location of at least one external fixation component of the external fixator.

9. The electronic device of claim 6, wherein the one or more inputs for determining the strut adjustment schedule includes a type of a strut attached to each external fixation component of the external fixator.

10. The electronic device of claim 9, wherein the one or more inputs for determining the strut adjustment schedule includes a length of the strut.

11. The electronic device of claim 10, wherein the length of the strut is received through user manipulation of a user interface of the electronic device.

12. The electronic device of claim 10, wherein the length of the strut is received automatically from a tracking system attached to the external fixator.

13. The electronic device of claim 12, wherein the length of the strut is received wirelessly from the tracking system attached to the external fixator.

14. The electronic device of claim 1, wherein the additional data related to the surgical procedure includes at least one of textual data and visual data.

15. A tracking system, comprising:
a first tracking component configured to be coupled to a first external fixation component of an external fixator; and
a second tracking component configured to be coupled to a second external fixation component of the external fixator, wherein the first tracking component comprises a controller, the controller to determine, in real-time, position data indicating a relative position between the first and second external fixation components based on data from the first tracking component, the controller to wirelessly transmit the determined position data to a remote device.

16. The tracking system of claim 15, the first tracking component to comprise an optical sensor.

17. The tracking system of claim 16, the optical sensor to comprise an optical camera.

18. The tracking system of claim 17, the second tracking component to comprise an LED target.

19. The tracking system of claim 15, the controller to wirelessly transmit the determined position data to the remote device during a surgical procedure for installing the external fixator on a patient.

20. The tracking system of claim 19, the determined position data used to determine the position for the second external fixation component during the surgical procedure.

21. The tracking system of claim 19, the determined position data used to determine a length and a type of a strut to attach to the first and second external fixation components during the surgical procedure.

22. The tracking system of claim 15, the controller to determine a length of a strut attached to the first and second external fixation components based on the determined position data after completion of the surgical procedure.

23. The tracking system of claim 22, the controller to wirelessly transmit the determined length of the strut to the remote device after completion of the surgical procedure.

24. The tracking system of claim 23, the determined length of the strut used to verify compliance with a strut adjustment schedule associated with the external fixator.
